(19) United States
(12) Patent Application Publication
(10) Pub. No.: US 2018/0338680 A1
(43) Pub. Date: Nov. 29, 2018

(54) Portable Eye Pressure Sensor

(71) Applicant: Jeffrey E. Koziol, Arlington Heights, IL (US)

(72) Inventor: Jeffrey E. Koziol, Arlington Heights, IL (US)

(21) Appl. No.: 15/986,953

(22) Filed: May 23, 2018

Related U.S. Application Data
(60) Provisional application No. 62/509,888, filed on May 23, 2017.

(51) Int. Cl. A61B 3/16

(52) U.S. Cl. CPC A61B 3/16

(57) ABSTRACT

Systems and methods for measuring and recording instantaneous or historic pressure topography or wave on the eye and the firmness of the eye where measuring IOP including a sensing section comprise two sensors, a microprocessor, a sensor support, and a handle. A sensor comprises contact-sensitive surface that makes contact with the eye during measuring procedure to determine area of the eye surface in contacted with the sensing section. Another second sensor comprises a force detector to determine the force applied by the eye surface when contacted by the sensing section. A microprocessor can receive essentially simultaneous input from the two sensors and/or in conjunction with two sensors can output time-tagged data that can be correlated to determine measured contact surface area and applied force at any given time. Data taken at various frequencies over a time period can be interpolated and/or extrapolated to facilitate correlation of measurements.

This application claims priority to prior U.S. Provisional Patent Application No. 62/509,888, filed May 23, 2017, the entire content of which is incorporated herein by reference.

### BACKGROUND OF THE DISCLOSURE

### 1. Field of Disclosure

Generally, exemplary embodiments of the present disclosure relate to the field of devices for ophthalmology, and in particular tonometry or tests to measure pressure inside an eye referred to as intraocular pressure (TOP). Exemplary implementations of certain embodiments of the present disclosure provide systems and methods for measuring eye pressure or tonometry and further provide a novel portable or hand held eye pressure sensor or tonometer.

### 2. Discussion of the Background of the Disclosure

Conventional methods for measuring IOP include Goldman tonometry, non-contact tonometry (NCT), electronic tonometry, and Schiotz tonometry, as generally described in “How Does Tonometry Eye Pressure Test Work?” by Troy Bedinghaus, OD (September 2016) attached hereto and made part of this disclosure as Appendix A (see also “Goldman Applanation Tonometry” by eyetec.net (opthalmictechnician.org 2015) attached hereto and made part of this disclosure as Appendix B).

All of these conventional tonometry techniques have various drawbacks. For example, NCT or “air puff” test can be inaccurate. Typically measurements from three “puffs” are averaged. However, the patient may feel discomfort and pull away from the machine during the air puffs, thus varying the distance from machine to eye surface which impacts the measurement accuracy. While Goldman tonometry is considered to be more accurate than NCT, it is much more invasive requiring anesthetic drops and fluorescein dye instilled into the eyes, and a probe that applies pressure on the cornea. Unlike Goldman tonometer which is not portable, electronic tonometry provides a handheld tonometer that looks like a pen, but like Goldman tonometer requires direct application to the cornea and is not considered as reliable as Goldman tonometry. Schiotz tonometry uses as indentation tonometer which determines pressure by measuring the depth of deformity caused by a small metal plunger applied directly to the cornea.

Presently, clinical methods that do not rely on instruments, for example when instruments are not available, allow patients to keep their eyes closed such that a skilled physician uses the thumb and index finger to ballot the eye and pick up a high pressure by touch.

A conventional tonometer that can measure IOP though the eyelid is described in “Transpalpebral Tonometer for Intraocular Pressure Measuring,” by A. P. Nesterov at www.diaton-tonometer.com/products/tonometer-diaton/articles (2017) attached hereto and made part of this disclosure as Appendix C. However, when using this tonometer the position with respect to the eye is critical, because it relies on “dynamic (ballistic) way of dosated mechanical influence on the eye for evaluating its elastic peculiarities” (see Id.), and any deviation from required position can cause erroneous results.

### SUMMARY OF THE DISCLOSURE

Exemplary embodiments of the present disclosure address at least such drawbacks by providing systems and methods including an implementation where a patient's eyelids are closed and a hand held instrument has at least two sensors in contact with the eye at the same time such that instantaneous or historic pressure topography or wave on the eye and the firmness of the eye can be measured and recorded.

An exemplary embodiment of the present disclosure provides a device for measuring IOP including a sensing section comprising at least first and second sensors, a microprocessor, a sensor support, and a handle. The first sensor comprises a contact-sensitive surface that makes contact with the eye during the measuring procedure to determine the area of the eye surface in contacted with the sensing section. The second sensor comprises a force detector to determine the force applied by the eye surface when contacted by the sensing section.

According to an exemplary implementation, a microprocessor, for example disposed in the handle of the device, can receive essentially simultaneous input from the first and second sensors. Alternatively, or in conjunction with, first and second sensors can output time-tagged data that can be correlated to determine measured contact surface area and applied force at any given time. In yet another implementation, data taken at various frequencies over a time period can be interpolated and/or extrapolated to facilitate correlation of measurements as needed.

According to another exemplary embodiment of the present disclosure, a device for measuring IOP can also include an internal memory for storing measured data obtained by the first and second sensors.

According to an exemplary implementation, a device for measuring IOP can include a wired or wireless transmitter for outputting data obtained by the first and second sensors essentially in real time, or on demand, for example in batches at certain pre-set intervals or on command.

According to yet another exemplary embodiment of the present disclosure, an IOP measuring system and method can include an IOP measuring device, data storage internal to the device, or external. for storing instantaneous and/or historic data obtained by the IOP measuring device, and internal or external display system for visual output, for example in a graphical format, of processed real time and/or historical data obtained by first and second sensors.

According to still further exemplary embodiment of the present disclosure, an IOP measuring device, system, or method provide a sensing section comprising a plurality of contact-sensitive sub-subsections and a plurality of force-sensing sub-section. A microprocessor (internal and/or external to the device, in direct, wired and/or wireless communication with the sensing section and/or with an internal and/or external memory storing data obtained by the sensing section) can be configured to process measured data and output, for example a 3D or color-coded graph to show IOP pressure over the eye surface in contact with the sensing section.

According to an exemplary implementation of the present disclosure, depending on the type and number of contact sensors and force sensors employed in the sensing section, a method for determining IOP can include any or all of: normalization of collected measured data to obtain a single value for the IOP measurement; generation of a two-dimensional graphical representation of IOP versus contact area; generation of a surface map or 3D graph of pressure across the eye surface in contact with the sensing section. As described with reference to other embodiments, a desired visual graphic or numeric output of raw or processed measures data obtained by sensing section can be performed in real-time and/or as post processing of historic data. In exemplary implementation, the output can be continuous so as to show in real-time, and/or historically, changes in the measurements as a function of time.

In yet further exemplary implementations of the embodiments of the present disclosure, evaluation of the results of IOP measurements can be performed with reference to a predetermined standard value, graph, or map of pressure value and/or values. Alternatively and/or in conjunction with comparison to a predetermined standard, patient's own historical data obtained by IOP measurements according to embodiments of the present disclosure can be used as a reference, or to create a patient's baseline, to evaluate the IOP measurements. In still further exemplary implementation, any such evaluation can be performed essentially in real time as IOP measurements are obtained and/or during post-processing of measured data.

Systems, methods and IOP measuring devices provided according to exemplary embodiments of the present disclosure can perform IOP measurement by direct contact of sensing section to eye cornea, or by contact of sensing section to the eyelid thereby avoiding discomfort of most conventional IOP measuring devices and techniques.

Furthermore, according to embodiments of the present disclosure, since the contact surface area is also considered and evaluated as part of the measuring process, position of IOP measuring device on the eye surface is taken into account.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein

FIG. 1A is an illustrative conceptual diagram showing diagrammatic representation of IOP measuring device according to an exemplary implementation of exemplary embodiments of the present disclosure with respect to a human eye.

FIG. 1B is an illustrative conceptual diagram showing diagrammatic representation of IOP measuring device according to another exemplary implementation of exemplary embodiments of the present disclosure.

FIGS. 2A, 2B, 2C, 3A, 3B, 3C, 4A, 4B, and 4C provide a diagrammatic illustration of application of a sensing section according to exemplary implementations of exemplary embodiments of the present disclosure to a surface of an eye to perform an IOP measurement procedure, and an exemplary representation of output data or information from IOP measuring device according to exemplary implementations of exemplary embodiments of the present disclosure.

FIG. 5 is another illustrative example of information and/or data collected and/or computed and output and/or displayed according to exemplary implementations of exemplary embodiments of the present disclosure.

FIG. 6 is an illustrative block diagram illustrating showing a diagrammatic representation of a system according to an exemplary embodiment of the present disclosure including an IOP measuring device according to exemplary implementations of exemplary embodiments of the present disclosure.

FIGS. 7A and 7B are illustrative conceptual diagram showing diagrammatic representation of an IOP measuring device according to yet another exemplary implementation of exemplary embodiments of the present disclosure.

### DETAILED DESCRIPTION OF EXEMPLARY EMBODIMENTS

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the described disclosure. Also, descriptions of well-known functions and constructions are omitted for clarity and conciseness.

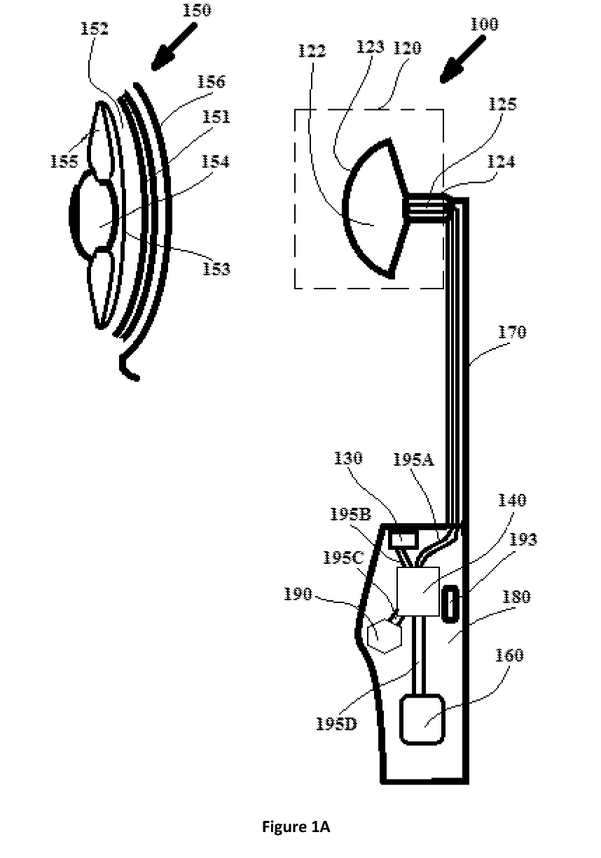

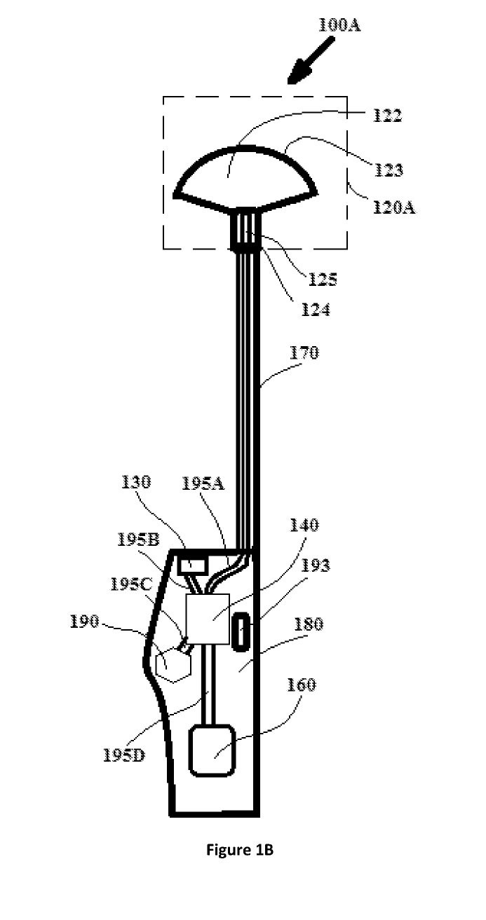

FIG. 1A (see also FIG. 1B) is a block diagram showing diagrammatic representation of IOP measuring device 100 according to exemplary embodiments of the present disclosure with respect to a human eye 150, whose well known anatomy includes cornea 151, anterior chamber 152, iris 153, pupil 154, and posterior chamber 155. Eye lid 156 is also shown since embodiments of the present disclosure provide for IOP measurement by direct contact with exterior surface of cornea 151 and/or by contact with exterior surface of eye lid 156.

Referring to an example of FIG. 1A, exemplary implementations of embodiments of the present disclosure provide an IOP measuring device 100 that includes a sensing section 120 with a first sensor 122 and a second sensor 124, a controller 130, a microprocessor 140, input/output (I/O) device(s) 160 such as wired and/or wireless transceiver and/or one or more communication port(s), a sensor support 170, and a handle 180. The first sensor 122 comprises a contact-sensitive surface 123 that makes contact with the eye 150 during the IOP measuring procedure, as explained in more detail below, to determine the area of the eye surface in contacted with the sensing section 120. The second sensor 124 comprises a force detector 125 to determine the force applied by the eye surface when contacting the eye by the sensing section 120. Similarly, FIG. 1B is a block diagram showing a diagrammatic representation of IOP measuring device 100A according to another exemplary embodiments of the present disclosure which includes section 120A in an essentially linear configuration with sensor support 170 and handle 180 (instead of an essentially 90-degree configuration of FIG. 1A). In the description that follows, any reference to a device having an essentially 90-degree configuration (as in FIG. 1A) is likewise applicable to a device having an essentially linear configuration (as in FIG. 1B).

IOP measuring device 100 can also include memory 190, which can be internal or external to microprocessor 140. Memory 190 can also comprise a portable removable memory such as USB or a flash drive. Any and/or all communication, such as 195A, 195B, 195C, and 195D, between any and/or all electronic components of IOP measuring device can be wired or wireless depending on the configuration of respective devices and other factors such as cost, portability, reliability, etc.

Power to various components, such as microprocessor 140 and/or I/O device(s) can be provide by an internal or external power source 193 which can include, for example, a battery (disposable or rechargeable).

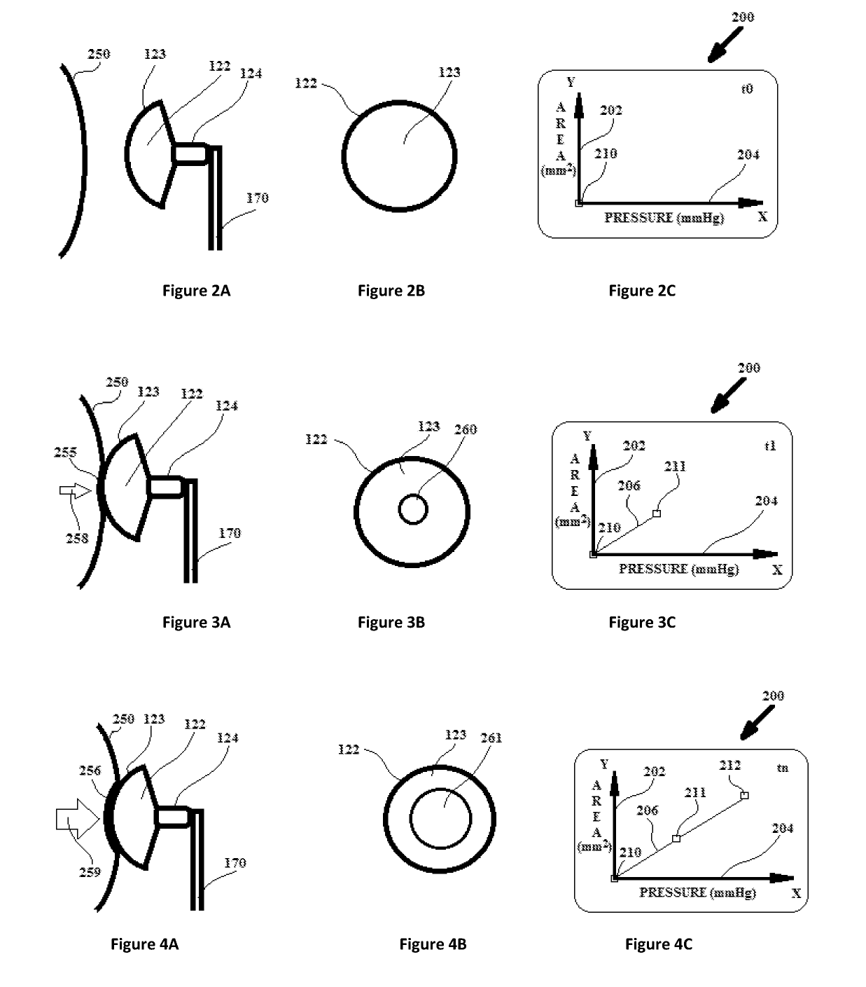

Methods of performing IOP measurements and operation of IOP measuring device and systems according to exemplary embodiments of the present disclosure are described with reference to FIGS. 2A-2C, 3A-3C and 4A-4C which provide a diagrammatic illustration of application of a sensing section 120 to a surface 250 of an eye 150 to perform an IOP measurement procedure, and an exemplary representation of output data or information from IOP measuring device 100. As note previously, according to embodiments of the present disclosure, surface 250 can be an exterior surface of cornea 151 or eye lid 156. While contact area of surface 250 is illustrated as being essentially circular, any shape of the contact area is within the scope of the present disclosure.

Referring to FIGS. 2A-2C, 3A-3C and 4A-4C, data output 200 of sensing section 120 according to an exemplary implementation can be represented in two-dimensional, X-Y axis, plot 206 of contact area (Y-axis) 202 for example in units of square millimeter (mm²) versus pressure (X-axis) 204 for example in units of millimeter mercury (mmHg). FIGS. 2A, 3A, and 4A provide a diagrammatical illustration of a side view of sensing section 120 of IOP measuring device 100 with respect to eye surface 250 of eye 150. FIGS. 2B, 3B, and 4B provide a diagrammatical illustration of contact-sensitive surface 123 of first sensor 122 from the perspective of the eye 150. FIG. 2C, 3C and 4C show an exemplary output 200 of IOP measuring device 100 before or during the IOP measuring process according to exemplary embodiments of the present disclosure.

In an exemplary implementation, a contact area 260,262, or 255,256, can be calculate based on interaction with eye surface 250 sensed by a contact-sensitive surface 123 of first sensor 122 at a time t, and pressure can be calculated based on force 258, 259 applied by eye surface 250 sensed by force detector 125 of second sensor 124 at the same time t. In an exemplary implementation, these calculations can be performed by microprocessor 140 and stored in memory 190 for real time output during the IOP measuring procedure, or historic download during or after IOP measuring procedure, via I/O device 160. In yet further exemplary implementation, output, activation of components, and other functions such as ON/OFF, can be controlled by a controller 130 which can include an interactive interface (such as simple switches and/or complicated touch screen displays) for receiving input from the user of IOP measuring device 100 and providing visual, audible, and or tactile output to the user. In still further exemplary implementation, controller 130 can receive and process external commands for example via wired or wireless communication with a user station (such a desktop, laptop, or personal display device (PDA)) 610, as illustrated in FIG. 6.

Referring to FIGS. 2A, 2B and 2C, prior to application of sensing section 120 to eye 150 (for example at time t0) data output 200 is illustrative 210 of no contact between sensing section 120, in particular contact surface 123 of first sensor 122, and the eye surface 250. As the IOP measuring device 100 indents the eye 150 at time t1 during the IOP measuring procedure, as shown in the example of FIGS. 3A, 3B and 3C, data output 200 is illustrative 211 of (1) Y-Axis contact area value—based on contact occurring over a portion 260 of contact-sensitive surface 123 at time t1 between sensing section 120 and portion 255 of eye surface 250, and (2) X-Axis pressure value—based on force 258 applied over portion 255 of eye surface 250 corresponding to portion 260 of contact-sensitive surface 123. As the IOP measuring device 100 further indents the eye 150 at time tn during the IOP measuring procedure, as shown in the example of FIGS. 4A, 4B and 4C, data output 200 is illustrative 212 of (1) Y-Axis contact area value increasing—based on increased indentation of eye surface 250 resulting in increased contact occurring over a portion 261 of contact-sensitive surface 123 at time tn between sensing section 120 and portion 256 of eye surface 250, and (2) X-Axis pressure value increasing—based on increased force 259 applied over portion 256 of eye surface 250 corresponding to portion 261 of contact-sensitive surface 123. The pressure that is exerted by application of IOP measuring device 100 to indent a given area of the cornea correlates with the IOP pressure.

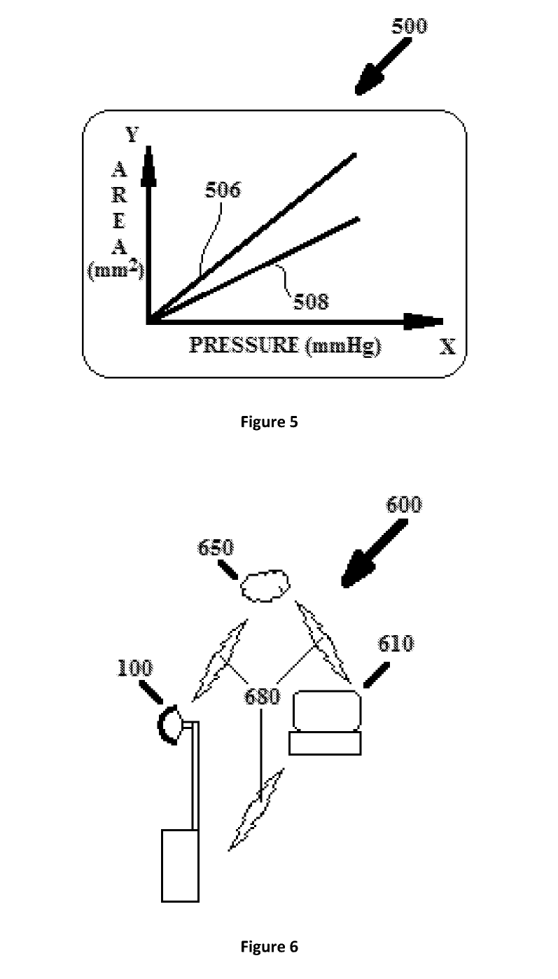

In an exemplary implementation of the present disclosure, IOP measurements taken during a procedure would produce a unique graph or data for the eye undergoing the IOP measuring procedure. Referring to FIG. 5, such measurement could be compared and evaluated with respect to other measurements, or a baseline, as illustrated by measurements taken during two IOP measuring procedures 506 and 508 (as noted, in an exemplary implementation graph 508 could be a baseline graph) where graph 506 may be illustrative of an eye with diagnosed IOP pressure of 20 mmHg, while graph 508 may be illustrative of an eye with diagnosed IOP pressure of 25 mmHg. A softer eye would have a large area indent (or interacting with contact-sensitive surface 123) for a given pressure that a firm eye.

FIG. 6 is a block diagram illustrating an exemplary embodiment of the present disclosure providing a system 600 including IOP measuring device 100 in wired and/or wireless (e.g., intra- or internet based) communication 680 with: external work station 610, which can provide and receive control information to/from device 100, perform data processing and/or display and/or storage; and/or external data storage 650, which could be cloud-based, shared and/or secure. Likewise, work station 610 can be in wired and/or wireless (e.g., intra- or internet based) communication 680 with data storage 650.

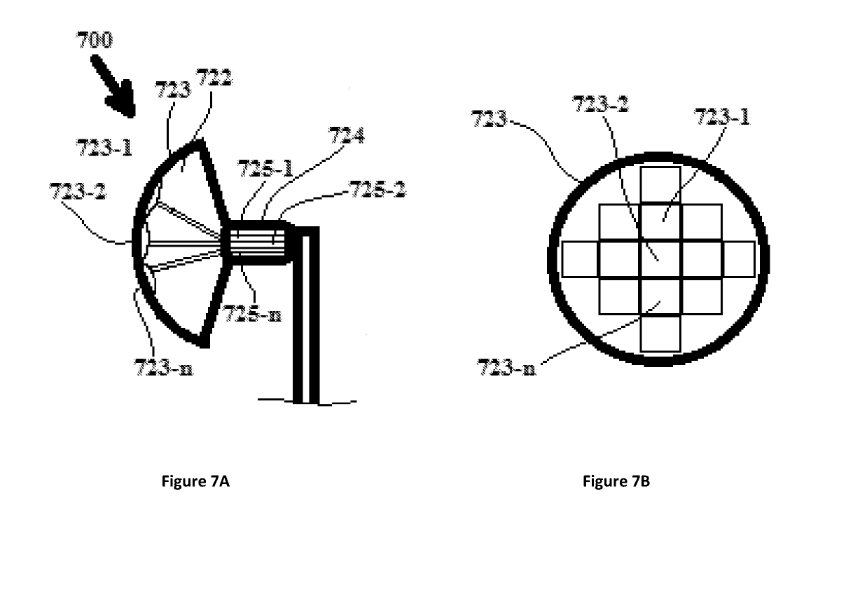

In yet another exemplary embodiment of the present disclosure illustrated in FIGS. 7A and 7B, IOP measuring device 700 can include a first sensor 722 comprising a contact-sensitive surface 723 with multiple contact-sensitive sub-areas 723-1, 723-2, . . . 723-n configured to sense corresponding contact pressure in conjunction with a second sensor 724 comprising a corresponding plurality of force detectors 725-1, 725-2, . . . 725-n detecting the force applied to the eye surface at each of the corresponding contact-sensitive sub-areas 723-1, 723-2, . . . 723-n. These measurements can be processed by an internal microprocessor of IOP measuring device 700, or by an external desk top such that of system 600 to compute a single, (for example normalized based on measurements from all sub-areas) value of pressure at time t of the IOP measurement, or produce a topological graph based on pressure values sensed in all sub-areas over contact sensitive surface. In an exemplary implementation, such graph could also be a 3D graph of pressure (Z-axis) with respect to a given contact-sensitive sub-area location (X-Y axis). The resolution of such a graphical representation would be directly related to the number of contact-sensitive sub-areas provided on contact-sensitive surface 723.

In yet another exemplary implementation, all or any portion of the measured data can be interpolated or extrapolated to produce a smoother graphical representation.

While the present disclosure has been shown and described with reference to certain exemplary embodiments thereof, it will be understood by those skilled in the art that various changes in form and details may be made therein without departing from the spirit and scope of the present disclosure.

Other objects, advantages and salient features of the disclosure will become apparent to those skilled in the art from the details provided, which, taken in conjunction with the annexed drawing figures, disclose exemplary embodiments of the disclosure.

Those of skill in the art further appreciate that the various illustrative logical blocks, modules, circuits, and algorithm steps described in connection with the embodiments disclosed herein may be implemented as electronic hardware, computer software, or combinations of both. Skilled artisans may implement the described functionality in varying ways for each particular application, but such implementation decisions should not be interpreted as causing a departure from the scope of the present disclosure. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

The above-presented description and figures are intended by way of example only and are not intended to limit the illustrative embodiments in any way except as set forth in the appended claims. It is particularly noted that various technical aspects of the various elements of the various exemplary embodiments that have been described above can be combined in numerous other ways, all of which are considered to be within the scope of the disclosure.

Accordingly, although exemplary embodiments have been disclosed for illustrative purposes, those skilled in the art will appreciate that various modifications, additions, and substitutions are possible. Therefore, the disclosure is not limited to the above-described embodiments, but may be modified within the scope of appended claims, along with their full scope of equivalents.

I claim:

1. A device for measuring IOP pressure comprising:
a sensing section comprising at least first and second sensors; and
a microprocessor in communication with said sensing section,
wherein
said first sensor comprises a contact-sensitive surface that makes contact with the eye during a measuring procedure to determine an area of a surface of an eye in contacted with said sensing section, and
said second sensor comprises a force detector to determine a force applied by the eye surface when contacted by the sensing section.

2. The device of claim 1, further comprising:
a sensor support to facilitate proper placement of said sensing section with respect to an eye; and
a handle connected to said sensor support, said handle comprises a housing, said microprocessor being disposed in said housing.

3. The device of claim 1, wherein said microprocessor receives essentially simultaneous input from the first and second sensors.

4. The device of claim 1, wherein said first and second sensors output time-tagged data.

5. The device of claim 4, wherein said microprocessor correlates said time-tagged data to determine measured contact surface area and applied force at a given time.

6. The device of claim 4, wherein said microprocessor interpolates and/or extrapolates said time-tagged data taken at various frequencies over a time period.

7. The device of claim 1, further comprising a non-transient computer-readable memory for storing measured data obtained by the first and second sensors and/or processed data output by said microprocessor.

8. The device of claim 1, further comprising a wired or wireless transceiver for outputting data obtained by the first and second sensors essentially in real time and/or on demand at certain pre-set intervals or on command.

9. A system comprising:
the device for measuring IOP pressure as claimed in claim 1;
data storage for storing instantaneous and/or historic data obtained by the IOP measuring device; and
an interface system for generating visual, tactile, and/or audio output indicative of processed in real time and/or historical data obtained by said first and second sensors of said IOP measuring device

10. The device of claim 1, wherein said sensing section comprising a plurality of contact-sensitive sub-subsections and a plurality of corresponding force-sensing sub-section.

11. The device of claim 10, wherein said microprocessor is configure as internal and/or external to the device and is in wired and/or wireless communication with the sensing section and/or with an internal and/or external memory storing data obtained by the sensing section and or processed data output by said microprocessor.

12. The device of claim 11, wherein said microprocessor processes said data obtained by the sensing section and generates an output indicative of pressure over the eye surface in contact with the sensing section.

13. A method of preforming IOP measurement, the method comprising:
positioning the device for measuring IOP pressure as claimed in claim 1 with respect to an eye surface;
applying pressure to the eye surface by said sensing section; and
evaluating the results of IOP measurements output by said microprocessor of the device for measuring IOP pressure with reference to a predetermined standard value, graph, and/or map of pressure value and/or values.

14. The method of claim 13 further comprising:
storing the results of IOP measurements output by said microprocessor as historical data and/or as a patient's baseline information;
evaluating current results of IOP measurements output by said microprocessor with reference to said stored data.

15. The method of claim 13, wherein said applying pressure to the eye surface by said sensing section comprises any one of:
direct contact of the sensing section to outer surface of cornea of the eye; or
contact of the sensing section to an outer surface of eyelid of the eye.
